(19) United States
(12) Patent Application Publication
(10) Pub. No.: US 2006/0069390 A1
(43) Pub. Date: Mar. 30, 2006

(54) Bone fixation device

(76) Inventors: Robert Frigg, Bettlach (CH); Bruno Lang, Horriwil (CH)

Correspondence Address: JONES DAY, 222 EAST 41ST STREET, NEW YORK, NY, 10017-6702, US

(21) Appl. No.: 11/250,518

(22) Filed: Oct. 14, 2005

Related U.S. Application Data
(63) Continuation of application No. PCT/CH03/00253, filed on Apr. 15, 2003.

(51) Int. Cl. A61F 2/30

(52) U.S. Cl. 606/61

(57) ABSTRACT

A bone fixation device for use in, for example, spinal column surgery, includes at least two bone fixation elements that each have a passage and a portion to be anchored on or in the bone. The device also includes at least two longitudinal, flexible connecting members that can be inserted and fastened in the passages of two adjacent fixation elements. The device can flexibly adapt to a patient's anatomy and to desired surgical results during the implantation of the device, and can then be rendered entirely rigid by simply blocking the fixation elements relative to one another. In addition, use of the device in the surgical treatment of the spinal column is minimally invasive.

### CROSS REFERENCE TO RELATED APPLICATION

This is a continuation of International Patent Application No. PCT/CH2003/00253, filed Apr. 15, 2003, the entire contents of which are incorporated herein by reference thereto.

### TECHNICAL FIELD OF THE INVENTION

The invention relates to bone fixation devices. More particularly, the invention relates to bone fixation devices that have flexible connecting members clamped in bone fixation elements that result in a rigid structure.

### BACKGROUND OF THE INVENTION

Most known spinal column stabilization systems use rigid connecting rods to connect the elements anchored in a spinal column. These rigid connections make it possible to fixedly set the desired spinal column adjustments made by a surgeon. The disadvantage of such rigid connections lies in their use during a surgical procedure. Only in exceptional cases will the surgeon succeed in attaching a plurality of anchoring elements to the spinal column such that they can be connected to one another by means of a straight, rigid rod. Often, the rigid rod has to be shaped during the operation to match the form of the spinal column. Such shaping is time consuming and may lead to unwanted stress concentrations in the anchoring elements. In order to improve the handling of these systems during surgical procedures, anchoring elements have been developed with polyaxially pivotable rod connecting members. This pivotability, while facilitating the insertion of the rigid rod (because the rod does not need to be pre-shaped with the same degree of precision), results in the disadvantage of a requiring a large volume to be occupied by these polyaxial connecting members. Another disadvantage of such known solutions, regardless of whether ordinary or polyaxially pivotable anchoring elements are used, is that once the connection with the rigid rod has been made, the possibilities of making further spinal column corrections are very limited.

An example of a known spinal column bone fixation device has two pedicle screws that can be implanted respectively in two adjacent vertebral bodies and connected to each other by means of two rods. The two rods may be fastened by clamping them in closed channels formed in the heads of the pedicle screws. The disadvantage of this device is that the device will remain flexible when flexible rods are used, even after fastening the connecting rods, and thus will not be sufficiently rigid to maintain the desired spinal column adjustments.

Another known spinal column bone fixation device has a series of pedicle screws that can be implanted respectively in the vertebral bodies of the concerned segment of the spinal column. The pedicle screws are connected to each other by means of two rods, which may be fastened by clamping them in closed channels formed in the heads of the pedicle screws. This device has the same disadvantage as the previous known device, because subsequent to the fixation of the cable in the heads of the pedicle screws, the entire assembly will still remain flexible and will thus lack the necessary rigidity.

Therefore, known flexible connecting members shaped in the form of thin wires, thin rods, or special flexible cables extending between the anchoring elements, all have the disadvantage of not being able—except for adjustments in the direction of tensile strain—to satisfactorily secure (i.e., hold in place) a correction made by the surgeon. It is therefore not possible to use the known flexible rod systems to relieve the spinal column, make positional corrections, or stabilize bone fractures.

### SUMMARY OF THE INVENTION

It is accordingly an object of the invention to create a bone fixation device which, on the one hand, is capable of flexibly adapting to a patient's anatomy and to the desired surgical results during the implantation of the device and, on the other hand, is capable of becoming completely rigid by simply blocking the elements of the device (i.e., rendering them substantially, if not completely, immovable) relative to one another. An additional objective is to achieve a minimally invasive treatment of the spinal column.

In one embodiment of the invention, a bone fixation device includes at least two fixation elements each having a passage and an anchoring portion operative to be anchored on or in a bone. The device also includes at least two longitudinal, flexible connecting members configured to be inserted and fastened in the passages of two adjacent ones of the bone fixation elements. The bone fixation device has a plane of symmetry, and the connecting members are arranged parallel to the plane of symmetry.

The advantages of the present invention make it possible to dispense with the complicated and time-consuming activity of adapting the rigid longitudinal support members to a patient's anatomy—without having to accept the disadvantages of using flexible longitudinal support members. In addition, once the bone fixation elements on the spinal column have successfully been connected, it is still possible to modify the position of the individual bone fixation elements relative to one another. The new positions of the concerned vertebral bodies relative to one another (spinal column correction) may be kept in place by blocking the flexible connecting members within the bone fixation elements. The elasticity of the flexible connecting members permits them to be inserted through an operative approach limiting soft-tissue injury to a minimum.

In an embodiment of the invention, the bone fixation elements are provided with a blocking element by means of which the mobility of connecting members inserted in their passage may optionally be fixed and released. By blocking the flexible connecting members in the passages of two adjacent bone fixation elements, their displaceability within the passages is inhibited, which results in conveying rigidity to the whole device.

The bone fixation elements may be selected from the following group: bone screws, bone retractors, and bone plates.

In one embodiment, the passage is a peripherally closed channel. A closed channel has the advantage of making it possible to reduce the dimensions of the bone fixation elements and to simplify their design.

In another embodiment, the passage is a peripherally open channel, preferably in the form of a groove having a U-shaped profile. The U-shaped profile advantageously allows the flexible connecting members to be inserted therein via the opening of the U-shaped profile, so that they no longer need to be threaded through the individual holes of peripherally closed channels. Moreover, it is possible, once the flexible connecting members have been assembled, to attach an additional bone fixation element.

In a particular embodiment, the connecting members are preferably rod-like, i.e. provided with a round cross-sectional profile.

Preferably, the number of connecting members is four, five, six, or seven.

In another particular embodiment, the cross section of the passages and the cross section of the connecting members are adapted to each other in such a way that the connecting members are received by positive engagement in the passages. The cross section of the passages may be suitably polyhedral, preferably square, rectangular, or triangular. The polyhedral form of the passage profile has the advantage that the connecting members inserted therein will be disposed in a defined position. Thus, it is possible to predefine the maximum flexural strength of the connection of two connecting members and/or of the whole device in one direction.

In still another embodiment, the connecting members in the passage are in contact with one another. Because of this arrangement, the blocking of the device will inhibit the longitudinal displaceability and rotational mobility of the connecting members relative to one another, which results in conveying the desired rigidity to the whole device.

In yet another embodiment, the bone fixation elements are bone screws and the portion to be anchored within the bone is shaped in the form of a threaded screw shank, the bone screw having a screw head in which a passage is in the form of a U-shaped groove. The passage may be closed by means of a blocking element, which may be in the form of a cap, so that the displaceability of connecting members placed in the groove can be inhibited.

The surface of the flexible connecting members may be provided with a roughened or microstructured pattern. This preferably enhances the adhesion by friction between the connecting members.

The surface of the flexible connecting members may be alternatively provided with a macrostructured pattern. This will also result in an enhancement of the adhesion by friction between the connecting members.

The surface of the bone fixation element passage may be provided with a roughened or microstructured pattern. This will result in an enhancement of the adhesion by friction between the passage and the connecting members.

The invention also includes a bone fixation method that includes the following: anchoring a bone fixation element having a passage by means of its anchoring portion on or in each of a number of bones or bone fragments, inserting two or more longitudinal, flexible connecting members in the passages of two adjacent bone fixation elements; and closing the passages of the two adjacent bone fixation elements using a blocking element, by means of which the mobility of the connecting members inserted in the passage relative to the bone fixation element is inhibited, thus conveying rigidity to the whole device.

The advantages achievable by using this method are many and include:
deviations from the optimal position of the two bone fixation elements no longer present a problem because the flexible connecting members allow a wide range of variability (unlike the known rigid, longitudinal support members which, in a case like this, first have to be removed from the patient's body and then bent into the desired shape by the surgeon using special apparatus);
blocking of the connecting members inhibits their flexibility, which conveys rigidity to the whole device, thus the final state equals that obtained by using rigid, longitudinal support members; and
the position of the individual bone fixation elements relative to one another may be changed and fixed in a new position without having to remove the connecting members inserted therein.

### BRIEF DESCRIPTION OF THE DRAWINGS

The detailed description will be better understood in conjunction with the accompanying drawings, in which like reference characters represent like elements, as follows:

FIG. 1 is a perspective view of a device according to the invention that includes two pedicle screws and four flexible, longitudinal rods in a non-blocked, flexible state;

FIG. 2 is a cross section of the head of a pedicle screw of the device according to FIG. 1; and

FIG. 3 is a cross section of another embodiment of a pedicle screw head having a closed passage.

### DETAILED DESCRIPTION OF THE INVENTION

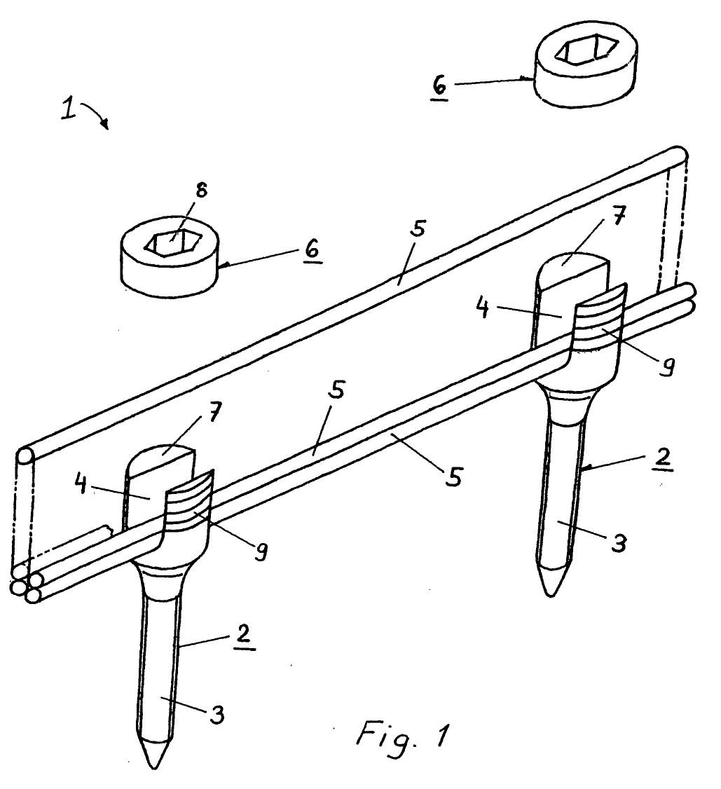

FIG. 1 shows a bone fixation device 1 that includes two bone fixation elements 2 (in the form of pedicle screws) and four longitudinal, flexible connecting members 5. Connecting members 5 are shaped in the form of thin, flexible rods that are connectable to bone fixation elements 2. Bone fixation elements 2 each have a portion 3 in the form of a threaded shank which may be anchored in the bone. Fixation elements 2 also have a head 7 having a U-shaped passage 4. Passages 4 of bone fixation elements 2 may be closed by means of a blocking element 6, which may be in the form of a hollow cap 6 having an internal screw thread 10 (see FIG. 2) and a hexagonal socket 8. The head 7 of each bone fixation element 2 has a corresponding external screw thread 9 to which the blocking element 6 may be screwed on in such a way that the longitudinal, flexible connecting members 5 inserted in passage 4 are fixed in their position both relative to one another and to the respective bone fixation element 2. This fixed state of the device 1 is represented in FIG. 2.

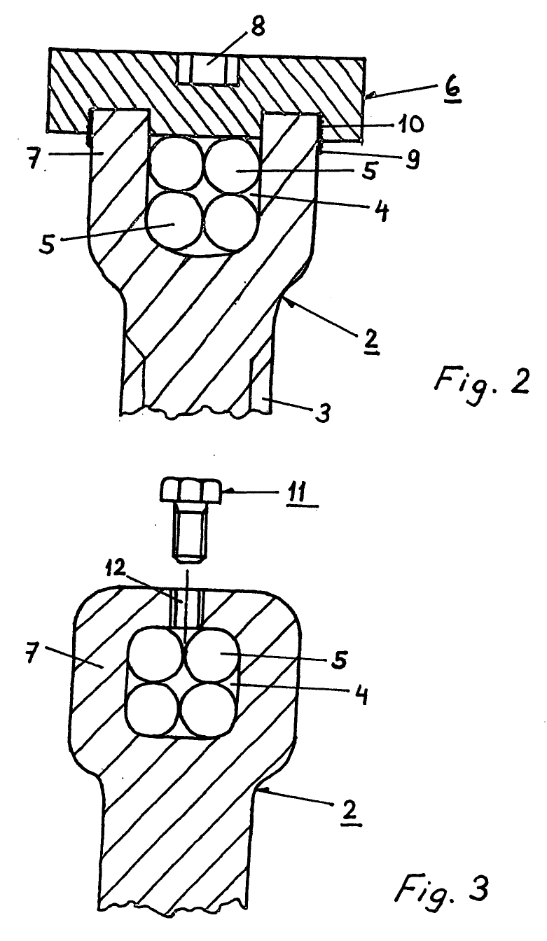

FIG. 3 shows a variation of bone fixation element 2, in which passage 4 is a closed, channel-like passage with an approximately square profile instead of the open, U-shaped passage of FIGS. 1 and 2. In order to firmly fasten the connecting members 5 inserted in passage 4, a locating screw 11 is provided which may be screwed in through a bore 12 until it exerts pressure on connecting members 5 so that they will be blocked.

1. A bone fixation device comprising:
at least two bone fixation elements, each having a passage and an anchoring portion operative to be anchored on or in the bone; and
at least two longitudinal, flexible connecting members configured to be inserted and fastened in the passages of two adjacent ones of the bone fixation elements; wherein:
the bone fixation device has a plane of symmetry, and the connecting members are arranged parallel to the plane of symmetry.

2. The device of claim 1 wherein the bone fixation elements are provided with a blocking element by means of which the mobility of connecting members inserted in the passage relative to the bone fixation element may optionally be fastened and released.

3. The device of claim 1 wherein the bone fixation elements are selected from among the following group: bone screws, bone retractors, and bone plates.

4. The device of claim 1 wherein the passage is a peripherally closed channel.

5. The device of claim 1 wherein the passage is a peripherally open channel in the form of a groove having a U-shaped profile.

6. The device of claim 1 wherein the connecting members are rod-like having a round cross-sectional profile.

7. The device of claim 1 wherein the number of connecting members ranges from four to seven.

8. The device of claim 1 wherein the diameter of the passages and the diameters of the connecting members correspond to one another such that the connecting members are received in the passages in positive engagement.

9. The device of claim 1 wherein the cross section of the passages is one of polyhedral, square, rectangular, or triangular.

10. The device of claim 1 wherein the connecting members inserted in the passage are in contact with one another.

11. The device of claim 1 wherein the bone fixation elements are bone screws and the portion to be anchored in the bone is a threaded screw shank, the bone screw having a head in which a passage is in the form of a U-shaped groove, the groove is configured to be closed by a blocking element in the form of a cap such that the displaceability of the connecting members placed in the groove is inhibited.

12. The device of claim 1 wherein the surface of the flexible connecting members has a roughened or microstructured pattern.

13. The device of claim 1 wherein the surface of the flexible connecting members has a macrostructured pattern.

14. The device of claim 1 wherein the surface of the passage has a roughened or microstructured pattern.

15. A bone fixation method comprising:
anchoring a bone fixation element on or in each of a number of bones or bone fragments via an anchoring portion of the bone fixation element, the bone fixation element also having a passage arranged symmetrically with respect to the anchoring portion;
inserting two or more longitudinal, flexible connecting members into the passages of two adjacent bone fixation elements; and
closing the passages of the two adjacent bone fixation elements using a blocking element to inhibit the mobility of the connecting members inserted into the passage relative to the bone fixation element, rendering the structure of the adjacent bone fixation elements and connecting members rigid.
